(19) United States
(12) Patent Application Publication
(10) Pub. No.: US 2016/0120898 A1
(43) Pub. Date: May 5, 2016

(54) METHODS AND COMPOSITIONS FOR THE DISRUPTION OF BIOFILMS AND TREATMENT OF DISORDERS CHARACTERIZED BY THE PRESENCE OF BIOFILMS

(71) Applicant: BREATHE EASY LIMITED, Hamilton (NZ)

(72) Inventor: Robert ELLIOTT, Hamilton (NZ)

(73) Assignee: BREATHE EASY LIMITED, Wellington (NZ)

(21) Appl. No.: 14/893,038

(22) PCT Filed: May 23, 2014
(86) PCT No.: PCT/NZ2014/000095

§ 371 Date: Nov. 20, 2015

Related U.S. Application Data
(60) Provisional application No. 61/826,927, filed on May 23, 2013.

(51) Int. Cl. A61K 33/02; A61K 45/06; A61K 9/12; A61K 31/225

(52) U.S. Cl. CPC A61K 33/02; A61K 31/225; A61K 45/06; A61K 9/12; C12Y 301/21; C12Y 302/01166

(57) ABSTRACT

Methods and compositions are provided for the disruption of biofilms and the treatment of disorders characterized by the presence of biofilms and/or abnormally viscous and/or cohesive bodily secretions, such as mucus and sputum. Disorders that can be effectively treated using the disclosed compositions and methods include cystic fibrosis (CF), endocarditis, urinary tract infections, middle-ear infections, chronic sinusitis, gingivitis, periodontal disease, bronchiectasis, chronic obstructive pulmonary disease (COPD), asthma, bronchitis, neonatal meconium aspiration syndrome, smokers' cough, chronic tonsillitis, chronic vaginitis, and fungal or bacterial infections. The compositions, which contain an effective amount of trisodium citrate and ammonium chloride, may be administered alone or in combination with one or more known therapeutic agents.

### TECHNICAL FIELD OF THE INVENTION

The present invention relates to methods and compositions for the disruption of biofilms and treatment of disorders characterized by the presence of biofilms, including lung disorders, such as cystic fibrosis, and other disorders caused by, or characterized by the presence of, bacterial infections.

### BACKGROUND OF THE INVENTION

A biofilm is a group of adherent microbial cells embedded within a matrix of composed of extracellular DNA, proteins and polysaccharides produced by the microbial cells. Microbial cells growing in a biofilm are physiologically distinct from planktonic cells of the same organism, which are single cells that can float or swim in a liquid medium. Biofilms can form on living or non-living surfaces and are prevalent in natural, industrial and hospital settings. The exopolysaccharide matrix that holds the biofilm together protects the cells within the biofilm, resulting in increased resistance to anti-microbial reagents, such as antibiotics, and to detergents.

Biofilms are involved in a wide variety of microbial infections in the body, including infections in cystic fibrosis, endocarditis, urinary tract infections, middle-ear infections, chronic sinusitis, chronic tonsillitis, formation of dental plaque, gingivitis, periodontal disease, and infections of implanted devices, such as catheters, heart valves, intrauterine devices and joint prostheses. Studies have shown that sub-therapeutic levels of antibiotics can induce biofilm formation in some microbial infections,

Cystic fibrosis (CF) is an inherited chronic life-threatening disorder that affects the lungs and digestive system of about 70,000 children and adults worldwide. CF causes serious lung damage due to a persistent cycle of opportunistic microbial infection and inflammatory response, and may also induce gastrointestinal dysfunction, with resulting nutritional deficiencies. Microbial infections that are commonly seen in individuals with CF include Haemophilus influenzae, Staphylococcus aureus, Pseudomonas aeruginosa, Burkholderia cepacia Complex, and the black yeast Exophiala dermatitidis (also known as Wangiella dermatitidis). Respiratory infections are the major cause of morbidity and mortality in individuals with CF.

The underlying cause of CF is one of a number of inherited mutations in the gene encoding a chloride channel protein (the CF transmembrane conductance regulator) which regulates the normal movement of chloride ions across cell membranes and affects cells that produce mucus, sputum, sweat, saliva and digestive liquids. These secretions are normally thin and watery, and act as lubricants. However, in patients suffering from CF, lung function is severely comprised by the presence of thick, sticky, highly viscous tracheo-bronchial secretions, which clog the lungs and lead to recurrent infections.

Pseudomonas aeruginosa is an infectious pathogen that is found in the secretions of CF patients as well as in immuno-compromised individuals and burn patients. P. aeruginosa accounts for the majority of respiratory infections in CF patients, with P. aeruginosa strains isolated from CF patients typically being mucoid. Non-mucoid strains of P. aeruginosa are generally treatable with antimicrobials. However, mucoid strains of P. aeruginosa are much more difficult to treat and produce large quantities of the mucoid exopolysaccharide alginic acid (alginate), which appears to have several effects including interference with complement-mediated polymorphonuclear leukocyte (PMN) chemotaxis, reduction in nonopsonic phagocytosis by PMNs, resistance to bacterial killing and interference with effective antimicrobial penetration of bacterial cells. Biofilms composed of mucoid alginate and acellular debris, including extracellular host and bacterial DNA, are formed and become virtually impenetrable by anti-microbial agents. Alginate from P. aeruginosa can also form insoluble calcium salts, which contributes to the impermeability of the hydrogel.

In CF patients, highly viscous secretions and biofilms resulting from bacterial infection block lumens, ducts and passageways, particularly in the lungs and the pancreas. Respiratory failure can occur when passageways in the lungs are blocked by thick secretions that cannot be expectorated, and when oxygen exchange is substantially reduced. Similarly, thick secretions in the pancreas block the release and activity of pancreatic enzymes that help to digest fats and proteins, preventing the body from absorbing key vitamins. Effective treatment of bacterial infection by mucoid P. aeruginosa strains in CF patients has been elusive, at least in part because the thick secretions produced by the mucoid strains and the chronic bacterial infections associated with alginate biofilms block entry of both antimicrobials and elements of the patient's immune system. The difficulty of treatment has been exacerbated by the emergence of strains of Pseudomonas and species of Bordetella that are resistant to available antimicrobials.

There are five main causes of increased sputum viscosity and cohesiveness:
1) insufficient water secretion;
2) the presence of excess extracellular DNA due to cell death;
3) the presence of alginate;
4) the presence of excess mucopolysaccharide; and
5) the presence of insoluble calcium salts of DNA, alginate and/or mucopolysaccharide.

Pulmozyme® (dornase alfa or rhDNase), which was approved for treatment of CF in 1993, acts by reducing the presence of excess extracellular DNA arising from cellular death and subsequent gelling of the DNA, thereby reducing the viscosity of the sputum. Treatment with Pulmozyme® generally results in a reduction in the number and severity of pulmonary infections and improved lung function. It is a standard of treatment for CF patients with intractable infective exacerbations, and is generally taken by aerosol inhalation by mouth once or twice daily.

Alginate can be depolymerized to oligosaccharides by the enzyme alginate lyase (also referred to as “alginase”). Alginate lyase has been studied as an agent for modifying the course of pseudomonal infection caused by mucoid strains of P. aeruginosa. In studies of experimental endocarditis caused by mucoid P. aeruginosa, the co-administration of alginate lyase with an amikacin regimen appeared to be effective in removing the exopolysaccharide from the surface of mucoid pseudomonal cells and enhancing the clearance of mucoid pseudomonal strains from the infection foci (Bayer, A. S. et al., Effects of Alginase on the Natural History and Antibiotic Therapy of Experimental Endocarditis Caused by Mucoid Pseudomonas aeruginosa, Infection and Immunity, 60:3979-3985 (1992)). Alginase has also been shown to reduce CF sputum viscosity and enhance macrophage or antimicrobial killing of the organism in vitro (Eftekhar, F. and Speer, D., Alginase Treatment of Mucoid Pseudomonas aeruginosa Enhances Phagocytosis by Human Monocyte-Derived Macrophages, Infection and Immunity, 56:2788-2793 (1998)). However the use of alginase in humans causes a severe allergic reaction.

It has been suggested that glycosaminoglycan degrading enzymes, such as heparanase, could be used to reduce the amount of mucopolysaccharide and thereby reduce sputum viscosity (see, for example, U.S. Pat. Nos. 6,153,187 and 6,423,312). No clinical data is available on the use of heparanase to treat CF.

Gram-negative bacteria in general, and P. aeruginosa in particular, are susceptible to destruction in vitro by chelating agents such as edetate sodium (EDTA). Researchers found that the majority of Pseudomonas strains isolated from fifty patients with CF were susceptible to EDTA, with marked synergism observed between EDTA and most antibiotics tested (Wood, R. E. et al., The effect of EDTA and antibiotics on Pseudomonas aeruginosa isolated from cystic fibrosis patients: A new chemotherapeutic approach, in Sturgess J M (ed): Perspectives in Cystic Fibrosis: Proceedings From the Eighth International Cystic Fibrosis Congress; Toronto, Canadian Cystic Fibrosis Foundation, pp. 365-369 (1980)). However subsequent clinical studies administering a combination of aerosolized EDTA in combination with oral tetracycline did not show any improvement in lung infection, any modification in the clinical course of CF, or any change in the pulmonary flora in CF patients with chronic Pseudomonas lung infection (Brown J. et al., Edetate Sodium Aerosol in Pseudomonas Lung Infection in Cystic Fibrosis, Am. J. Dis. Child 139:836-9 (1985)). Furthermore, administration of EDTA by aerosol has been found to be irritating, and it has been suggested that EDTA may act as a bronchoconstrictor (Beasley et al., Br. Med. J., 294:1197-8 (1987)).

The level of calcium salts has been shown to be elevated in cells of tracheal mucosa and, to a lesser extent, mucus glands of CF patients. These calcium salts tend to appear as apatite-like crystals (Cantet et al., Virchows Arch. 439:683-90 (2001)). The presence of calcium in mucosa has been shown to increase inflammatory reactions (Ribeiro et al., J. Biol. Chem. 208:17798-806 (2005)). In addition, it is believed that lowering calcium levels in mucosa has beneficial effects on the chloride pump defect that is the hallmark of CF (Middleton et al., Am. J. Respir. Crit. Care Med. 168:1223-6 (2003)).

Current treatments for CF generally attempt to control infection through antimicrobial therapy and to promote mucus clearance using postural drainage and chest percussion. Effective and long-lasting treatments for CF and for reducing mucoid bacterial populations have been elusive and there remains a need for effective treatment of disorders characterized by the presence of abnormally viscous bodily secretions, such as sputum, and/or by the presence of biofilms.

### SUMMARY

The present invention provides methods and compositions for the treatment of disorders and/or microbial infections characterized by the presence of biofilms. The disclosed compositions and methods can also, or alternatively, be employed to reduce the viscosity and/or cohesiveness of mucus and/or sputum in a patient in need thereof and can be effectively employed in the treatment of disorders characterized by the presence of bodily secretions, such as sputum or mucus, having an abnormal or excessive viscosity, and/or characterized by the presence of excess amounts of mucus and/or sputum. Disorders that can be effectively treated employing the disclosed methods and compositions include, but are not limited to, cystic fibrosis (CF), endocarditis, urinary tract infections, middle-ear infections, chronic sinusitis, chronic tonsillitis, gingivitis, periodontal disease, bronchiectasis, chronic obstructive pulmonary disease (COPD), asthma, bronchitis, neonatal meconium aspiration syndrome, smokers' cough, chronic tonsillitis, chronic vaginitis, and fungal or bacterial infections, including infections of implanted devices, such as catheters, heart valves, intrauterine devices and joint prostheses. Microbial infections that can be treated using the disclosed methods and compositions include, but are not limited to, infection with Haemophilus spp., such as Haemophilus influenza; Staphylococcus spp., such as Staphylococcus aureus; Pseudomonas spp., such as Pseudomonas aeruginosa; Burkholderia cepacia Complex; Wangiella dermatitidis; Aspergillus spp.; and Candida spp.

In one aspect, compositions are provided that comprise, or consist essentially of, trisodium citrate and ammonium chloride. In certain embodiments, the disclosed compositions comprise trisodium citrate and ammonium chloride as the sole active ingredients. The trisodium citrate is generally present at a concentration between 10 mM to 80 mM or between 25 mM to 65 mM, and preferably at a concentration of 55 mM. The ammonium chloride is generally present at a concentration between 50 mM to 100 mM or between 60 mM to 90 mM, and preferably at a concentration of 75 mM. In certain embodiments, the trisodium and ammonium chloride are present in an amount effective to disrupt a microbial biofilm. In preferred embodiments, the disclosed compositions are isotonic and have a neutral pH (i.e. a pH of about 7.1). The trisodium citrate can also, and/or alternatively, be present in an amount effective to decrease the viscosity and/or cohesiveness of the mucus and/or sputum of a patient when administered to a patient in need thereof compared to prior to administration. In certain embodiments, the trisodium citrate is present in an amount effective to decrease the viscosity and/or cohesiveness of the mucus and/or sputum by at least 25-50%.

The present disclosure also provides formulations comprising a composition disclosed herein and a therapeutic agent. Therapeutic agents that can be effectively employed in such formulations include, but are not limited to, anti-microbial agents, antibiotics, DNase, alginase, ascorbic acid and heparanase.

In certain embodiments, the disclosed compositions and formulations are formulated for delivery to the respiratory tract, the gastrointestinal tract and/or the reproductive tract.

In another aspect, methods for treating a disorder characterized by the presence of biofilm are provided, such methods comprising administering a composition or formulation disclosed herein. The compositions and/or formulations can be administered simultaneously with, or sequentially to, one or more known therapeutic agents, such as anti-microbial agents, antibiotics, DNase, alginase, heparanase, ascorbic acid and antimicrobial agents. The disclosed compositions and/or formulations can be administered prior to administration of the known therapeutic, for example at least four hours prior to administration of the known therapeutic. Alternatively, the disclosed compositions and/or formulations can be administered concurrently with the known therapeutic provided there is no adverse interaction with the known therapeutic agent.

In one embodiment, the disclosed compositions are administered in either an aerosol form or in a dry powder form, and are delivered to a target site selected from the group consisting of: the respiratory tract, the gastrointestinal tract, and the reproductive tract.

### BRIEF DESCRIPTION OF THE FIGURES

FIG. 1 shows the viscosity of a sputum sample at 0 sec, 30 sec, 90 sec and 150 sec after addition of different volumes of 50mM sodium citrate.

### DETAILED DESCRIPTION

As noted above, the present disclosure provides compositions, formulations and methods for the treatment of a disorder characterized by the presence of a microbial biofilm. While not wishing to be held to theory, the inventor believes that the disclosed compositions and formulations thin secretions, enhance antibiotic activity against opportunistic bacterial and fungal pathogens, and diminish adverse immune reactions associated with biofilms.

The disclosed compositions, formulations and methods can also, or alternatively, be used to treat disorders in which abnormal or excessive viscosity and/or cohesiveness of one or more bodily secretions, such as mucus or sputum, is a symptom or cause of the disorder. Mucus or sputum that is abnormally or excessively viscous and/or cohesive has a viscosity or cohesiveness that is measurably more viscous or cohesive than mucus or sputum from a normal, healthy patient. Such mucus or sputum may cause discomfort in a patient and/or cause or exacerbate a disease in the patient. In particular embodiments, the disclosed compositions, formulations and methods are effectively employed in the treatment of cystic fibrosis (CF), endocarditis, urinary tract infections, middle-ear infections, chronic sinusitis, gingivitis, periodontal disease, bronchiectasis, chronic obstructive pulmonary disease (COPD), asthma, bronchitis, neonatal meconium aspiration syndrome, smokers' cough, and fungal or bacterial infections, including infections of implanted devices, such as catheters, heart valves, intrauterine devices and joint prostheses. In specific embodiments, the compositions, formulations and methods disclosed herein are employed to treat bacterial, fungal and/or viral infections of the lungs and respiratory tract, including bacterial pneumonia (for example caused by Streptococcus pneumonia); influenza (for example, H1N1); infection with Haemophilus influenzae, Staphylococcus aureus, Pseudomonas aeruginosa, Burkholderia cepacia Complex, Wangiella dermatitidis, Aspergillus spp. and/or Candida spp.; and other disorders characterized by the presence of biofilms and/or increased sputum production.

The inventor has surprisingly determined that a composition comprising trisodium citrate and ammonium chloride is highly effective both in increasing the susceptibly of bacterial infections to treatment with antibiotics and in the treatment of fungal infections, such as infection with Wangiella dermatitidis.

Both trisodium citrate and ammonium chloride are non-toxic and are well-tolerated by humans. As detailed below, the inventor has determined that sodium citrate is effective in reducing sputum viscosity, at least in vitro, is well tolerated when administered via nebulization, and is effective in the treatment of cystic fibrosis. While not wishing to be held by theory, the inventor believes that sodium citrate decreases the viscosity and/or cohesiveness of sputum and/or mucus by (a) converting insoluble calcium salts of alginate, extracellular DNA and mucopolysaccharides to soluble salts; (b) reducing the calcium content of tracheal secretions and thereby enhancing the defective chloride pump; and/or (c) reducing intracellular calcium. As noted above, there is some evidence that the presence of intracellular calcium crystals causes inflammation of the lung in patients with CF.

In certain embodiments, the disclosed compositions comprise trisodium citrate at a concentration of about 10 mM to about 80 mM, such as 10 mM, 15 mM, 20 mM, 25 mM, 30 mM, 35 mM, 40 mM, 45 mM, 50 mM, 55 mM, 60 mM, 65 mM, 70 mM, 75 mM or 80mM, and ammonium chloride at a concentration of about 50 mM to about 100mM, such as 50 mM, 55 mM, 50 mM, 65 mM, 70 mM, 75 mM, 80 mM, 85 mM, 90 mM, 95 mM, or 100 mM. In preferred embodiments, the trisodium citrate is present in an amount effective to reduce the viscosity and/or cohesiveness of sputum or mucus as compared to the viscosity and/or cohesiveness prior to contact with the composition. In certain embodiments, the disclosed compositions comprise trisodium citrate in an amount effective to reduce the viscosity of sputum or mucus by at least 20%, at least 25%, at least 30%, at least 35%, at least 40%, at least 45%, at least 50%, or at least 55%, compared to the viscosity prior to contact with the composition. Techniques for measuring the viscosity of sputum are well known in the art and include, for example, a pourability test, as described below and the use of a viscometer, such as those available from proRheo GmbH (Germany).

The compositions may additionally comprise one or more components selected from the group consisting of: pharmaceutically acceptable carriers, such as water, phosphate buffered saline, dextrose solution; preservatives; and the like. In specific embodiments, the trisodium citrate and ammonium chloride can be formulated as a more concentrated solution and then diluted in water to attain an isotonic solution. In one embodiment, the volume of administration is 5 ml in an adult patient with CF. The compositions can be administered to a patient in need thereof one to two times a day, or as needed.

In another embodiment, compositions are provided that consist essentially of trisodium citrate and ammonium chloride. As used herein, the term “composition consisting essentially of trisodium citrate and ammonium chloride” is used to indicate a composition in which trisodium citrate and ammonium chloride are the only components that are effective in disrupting microbial biofilm and/or reducing the viscosity of sputum by at least 20%, at least 25%, at least 30%, at least 35%, at least 40%, at least 45%, at least 50%, or at least 55%.

Methods for the treatment of a disorder in a subject are also provided, such methods comprising administering a composition or formulation disclosed herein to a patient in need thereof. As used herein, the terms “patient” and “subject” refer to a mammal, preferably a human, and are used interchangeably.

Preferably, the disclosed composition and/or formulation is administered to a patient in need thereof in an amount that is effective to disrupt the biofilm and/or provide a statistically significant increase in the liquefaction of the mucus and/or sputum. In certain embodiments, the amount of the composition or formulation administered to the patient is sufficient to result in a change in the liquefaction of the mucus or sputum by at least 20%, at least 25%, at least 30%, at least 35%, at least 40%, at least 45%, at least 50%, or at least 55%. Alternatively, the composition is administered in an amount sufficient to clear a blockage or inhibition of function caused by the mucus or sputum as indicated by an improvement in the forced expiratory volume in one second (FEV1) of at least 5%, at least 9% or at least 12% after prolonged administration compared with the pre-administration value.

Those of skill in the art will appreciate that the preferred dosing regimen can be varied depending on the route of administration, symptoms, body weight, health and condition of the patient and the like, and that the preferred dosing regimen can be readily determined using known techniques.

In certain aspects, the disclosed compositions are delivered to at least one or more target site(s) characterized by: (i) the presence of microbial biofilm; (ii) an accumulation of bodily secretions having an undesirably high viscosity and/or cohesiveness; and/or (iii) an undesirably high accumulation of secretions. Target sites may include passageways in the lungs, airways within and leading to the lungs, nasal passageways, gastro-intestinal lumens, and lumens and cavities in the pancreas, digestive organs or reproductive organs. In one aspect, the disclosed compositions are contacted with a bodily secretion, such as mucus or sputum, located in the respiratory tract, the gastrointestinal tract and/or the reproductive tract of a patient.

In other embodiments, the disclosed compositions are employed for the reduction of biofilms associated with gingivitis, periodontal disease and chronic tonsillitis, and are included in toothpaste, mouthwashes, solutions intended to be gargled, and the like.

Methods and routes for delivering compositions to target sites are well known to those of skill in the art. In certain embodiments, the disclosed compositions are delivered by inhalation or nasally, for example using a nebulizer or an aerosol, mist or vapor delivery system. In one embodiment, the compositions are formulated for delivery to the airways as mist or particles entrained in gaseous or liquid carriers using a nebulizer such as an ultrasonic nebulizer, electronic micropump, liquid projection apparatus or mist/vapor generating apparatus, which are well known in the art. The compositions are delivered preferably at zero or low velocity to the mouth or nose, preferably during the inspiratory cycle only.

Alternatively, the disclosed compositions can be formulated for delivery to the airways using dry particle delivery devices. For example, the disclosed compositions and formulations can be administered to the respiratory tract in the form of a dry powder by means of inhalation. In certain embodiments, powdered compositions and formulations are administered in an amount of about 5 mg to about 50 mg, about 10 mg to about 20 mg, or about 15 mg one or two times per day. Dry compositions and formulations can be administered either with or without an excipient, such as lactose. Various dry powder delivery devices that can be effectively employed to deliver dry compositions and formulations are well known to those skilled in the art. For example, dry compositions and formulations may be administered using a dry powder inhaler, such as those described in U.S. Pat. Nos. 6,209,538, 6,889,690, 7,617,822, 7,694,676 and 7,708,011.

In another aspect, the compositions and/or formulations disclosed herein are administered to a patient in combination with one or more known therapeutic agents currently employed in the treatment of microbial and/or fungal infections, and/or in the treatment of CF. For example, the disclosed compositions and formulations can be used in combination with anti-microbial agents, antibiotics, ascorbic acid, DNase (e.g., Pulmozyme®), heparanase, alginase and combinations thereof. The disclosed composition or formulation and the known therapeutic agent can be formulated together or separately, and can be administered at the same time, sequentially or at different times. For example, as DNase is typically formulated in a calcium-containing buffer, it is advantageous to separate the administration of DNase and a trisodium citrate-ammonium chloride composition by between 4-12 hours, such as 8 hours. Similarly, when a trisodium citrate-ammonium chloride composition is employed in combination with heparanse and/or alginase, the treatments can be separated by between 4-12 hours, such as 8 hours. In one embodiment, a composition disclosed herein is administered to a patient in need thereof approximately 8 hours prior to the administration of DNase. The DNase, heparanase, alginase and/or other known therapeutic agent are administered using standard dosage regimes known to those of skill in the art.

Administration of the disclosed compositions and formulations may be accompanied by co-administration with an antimicrobial agent effective in reducing P. aeruginosa and/or other bacterial or fungal populations, such as Zithromax™, Tobramycin, and the like. Co-administration of an antimicrobial agent may be at the same time, sequentially or at a different time from administration of the composition, and the antimicrobial agent may be provided in the same formulation or in a different formulation.

The disclosed compositions and formulations can also be employed to reduce formation of biofilms on intravascular devices, such as catheters, by adding the compositions and/or formulations to known catheter lock solutions.

As another example, the disclosed compositions and formulations can also be employed to prevent and/or treat biofilms and infections in patients following maxillofacial surgery or trauma. In such an example, a convenient mode of administration may be ventilation (for instance using a nebulizer), as discussed above.

Those of skill in the art will appreciate that the compositions disclosed herein can effectively be employed to disrupt biofilms in other applications, including agricultural and/or horticultural applications. For example, the compositions disclosed herein can be safely employed to reduce the incidence of plant and crop diseases, such as fungal and bacterial diseases, characterized by the presence of biofilms such as, but not limited to, kiwi fruit vine canker, fungal grape rot, tomato blight, and onion rot. In one embodiment, the compositions disclosed herein can be applied, either simultaneously or sequentially, with salicylic acid to reduce the incidence of vine canker.

### EXAMPLES

### Example 1

The ability of a combination of trisodium citrate and ammonium chloride to treat bacterial and fungal infections in patients with cystic fibrosis was examined as follows.

### Study 1

The subject in this study was a 26 year old female with cystic fibrosis, diagnosed by newborn screening test and confirmed by sweat test and genetic analysis (delta F508 mutation). She had suffered frequent bouts of pulmonary infections associated with the growth of Pseudomonas aeruginosa since the age of seven. This organism was cultured every time a sputum sample was taken, despite frequent courses of oral and intravenous antibiotics, and, over the last decade, almost continuous Tobramycin delivered twice a day by nebulizer. The subject had also received courses of nebulized alpha-dornase to alleviate tenacious sputum, without much effect.

Before treatment with an isotonic combination of trisodium citrate and ammonium chloride began, her sputum again showed a heavy growth of mucoid Pseudomonas and the black fungus Wangiella dermatitidis, which she had shown on most sputum samples despite antifungal treatment. Her lung function had steadily deteriorated, being 58% of predicted normal value for FEV1. Her sputum was thick and very difficult to expectorate; it clung to hand basins and toilet bowls despite forcible flushing.

Administration of a combination of trisodium citrate at a concentration of 55 M and ammonium chloride at a concentration of 75 mM (5 ml nebulized twice a day), in addition to Tobramycin was commenced. Within hours there was notable thinning of sputum which subsequently maintained a thin watery consistency. Within two weeks of commencement of administration of trisodium citrate and ammonium chloride, her lung function rose to 76% of normal predicted value. Her sputum over the last three months of treatment did not grow mucoid Pseudomonas, but only on one occasion of three grew a scanty growth of non-mucoid Pseudomonas, and the Wangiella fungus disappeared. Six months after the start of treatment, the subject remained well and experienced no adverse effects from treatment with trisodium citrate and ammonium chloride.

The disappearance of both Pseudomonas and Wangiella is without precedent in both this subject and others with CF. The improvement in lung function (+18% FEV1) is greater than that found with alpha dornase (average 9%).

### Study 2

A sixty year old male volunteered to try the isotonic combination of trisodium citrate at a concentration of 55 M and ammonium chloride at a concentration of 75 mM described above. This patient had the rare inherited condition antitrypsin deficiency, which is characterized by progressive loss of lung function and infection with opportunistic bacteria.

For years he had grown Pseudomonas aeroginosa in his sputum despite frequent courses of antibiotics. Two years ago he sought a lung transplant as his lung function had deteriorated to the point where he required continuous extra oxygen, and was deemed suitable apart from the growth of Pseudomonas in his sputum, which is a contraindication for such surgery.

The patient was administered the trisodium citrate and ammonium chloride composition using a nebulizer, at a dose of 2.5-5 ml, twice a day for five weeks. He noted a lessening of sputum viscosity, and increase in cough. At the end of this period (January 2014), his sputum was culture negative for Pseudomonas. He had concurrently been taking an antibiotic which was previously ineffectual. In April 2014, his sputum again returned a negative culture for Pseudomonas which rendered him potentially eligible for lung transplantation.

### Example 2

The ability of isotonic trisodium citrate together with 4 mg/mL ammonium chloride (pH 7.14) to dissolve alginate beads was examined as follows.

2 mL alginate beads were prepared from Alginate 5710/10 (airflow 2.455, gelled in calcium chloride for 5 minutes, rinsed and washed with NaCl₂, size 600 um). 5 mL Isotonic sodium citrate was added to 2 mL alginate beads and incubated on roller for 2 minutes, rinsed and washed with sodium chloride. 5 mL Isotonic sodium citrate was added to 2 mL alginate beads and incubated on roller for 5 minutes, rinsed and washed with NaCl. 5 mL isotonic sodium citrate plus 4 mg/mL ammonium chloride (pH 7.14) was added to 2 mL alginate beads and incubated on roller for 2 minutes, rinsed and washed with NaCl. 5 mL isotonic sodium citrate plus 4 mg/mL ammonium chloride (pH 7.14) was added to 2 mL alginate beads and incubated on roller for 5 minutes, rinsed and washed with NaCl. All samples were analyzed via light microscopy and also by visual determination of alginate bead dissolution.

Table 1 below gives the solubility of the alginate beads immediately after the beads had been incubated with either isotonic sodium citrate or isotonic sodium citrate +4mg/mL ammonium chloride (pH 7.14) and rinsed and washed with NaCl.

TABLE 1
solubility of alginate beads

| | | Isotonic sodium |
|---|---|---|
| | Isotonic sodium | citrate + 4 mg/mL |
| Incubation Time | citrate | ammonium chloride (pH 7.14) |
| 2 min | Not dissolved | Not dissolved |
| 5 min | Not dissolved | Not dissolved |

In all conditions, the alginate beads did not immediately dissolve and alginate beads were still clearly visible, although they had swollen to 1000 um+, which made them difficult to see using light microscopy. However, in all cases, the alginate beads did eventually dissolve, but at varying times after the incubation with sodium citrate or sodium citrate +4 mg/mL ammonium chloride (pH 7.14) was complete.

After treatment, the tubes were monitored visually. The alginate beads that were incubated with 5 mL isotonic sodium citrate +4 mg/mL ammonium chloride (pH 7.14) for 2 minutes were completely dissolved within 7 minutes. This was much quicker than the alginate beads that were incubated with 5 mL isotonic sodium citrate for 2 minutes and were still dissolving after 30 minutes.

These results demonstrate that isotonic sodium citrate and isotonic sodium citrate +4 mg/mL ammonium chloride (pH 7.14) do not dissolve alginate beads within 2 minutes. Dissolution continues after incubation with sodium citrate has been completed. When alginate beads were incubated with isotonic sodium citrate +4 mg/mL ammonium chloride (pH 7.14) for two minutes, complete dissolution of alginate was completed within 7 minutes post NaCl wash. Complete dissolution of alginate beads incubated with isotonic sodium citrate for 2 minutes was not complete after 30 minutes.

### Example 3

The ability of sodium citrate solution to decrease the viscosity of sputum samples was examined as follows.

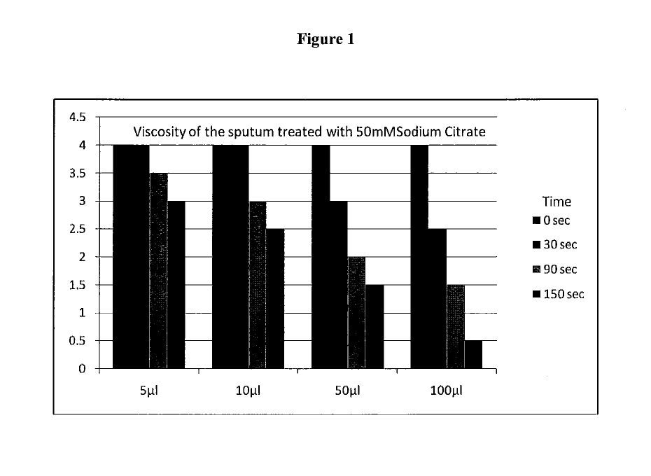

Three different sputum samples were employed: sample 1 was of a runny consistency; sample 2 was of an intermediate consistency; and sample 3 was of a heavy consistency. 100 ul samples were measured and transferred to assay tubes in duplicate (except for sample 1 which was in short supply) and placed in a 37° C. preheated Thermomixer. 10 ul of saline (0.9% NaCl) was used as a negative control. 5 ul, 10 ul, 50 ul or 100 ul of 55 mM aqueous sodium citrate solution was added per tube and mixed (400 rpm). Each tube was checked for pourability, or viscosity, at 30 sec, 90 sec and 150 sec by observing the movement of the sputum sample along the wall of the tube. The pourability of sample 3 was also checked at 5 minutes. The pourability was graded as follows: “0”=no change to “+++>” dissolved. The results of the study are shown below in Table 2 for samples 1-3, and in FIG. 1.

For sample 1, no change in its watery nature was seen at any addition of either saline or citrate at any time after incubation. For sample 2 (intermediate sputum viscosity) some decrease in viscosity was seen after 30 seconds of incubation with the larger additions of citrate. Sample 3, which contained the most viscous sputum, was more resistant to the effects of citrate and took five minutes before the full effects were seen.

TABLE 2

| | | TIME | | | | | | | | | | | | | | |
|---|---|---|---|---|---|---|---|---|---|---|---|---|---|---|---|---|
| | | 0 | | | 30 sec | | | 90 sec | | | 150 sec | | | 5 min | | |
| | | SAMPLE | | | | | | | | | | | | | | |
| REAGENT | VOL. | 1 | 2 | 3 | 1 | 2 | 3 | 1 | 2 | 3 | 1 | 2 | 3 | 1 | 2 | 3 |
| Saline | 10 ul | +++> | 0 | 0 | +++> | 0 | 0 | +++> | +/− | +/− | +++> | +/− | +/− | n/a | n/a | +/− |
| 55 mM | 5 ul | +++> | 0 | 0 | +++> | 0 | 0 | +++> | +/− | +/− | +++> | + | +/− | n/a | n/a | +> |
| Citrate | 10 ul | +++> | 0 | 0 | +++> | 0 | 0 | +++> | + | + | +++> | +> | + | n/a | n/a | ++> |
| | 50 ul | +++> | 0 | 0 | +++> | + | + | +++> | ++ | +> | +++> | ++> | +> | n/a | n/a | +++> |
| | 100 ul | n/a | 0 | 0 | n/a | + | + | n/a | ++> | ++ | n/a | +++> | ++ | n/a | n/a | +++> |

The results of this experiment demonstrate that sodium citrate at concentrations of 5 ul and 100 ul was the most effective, in terms of reducing sputum viscosity, and that the maximal effectiveness was achieved at 5 min following incubation.

### Example 4

The safety and toxicity of aqueous sodium citrate solution was examined as follows. Treatment solutions were prepared by diluting 0 mM, 11 mM and 22 mM sodium citrate in 2 ml of water. The solutions were nebulized and administered to three healthy volunteers in successive treatments, with ascending sodium citrate dosage volumes and a 5 min break between each treatment. The forced expiratory volume in 1 sec (FEV1) and forced vital capacity (FVC) were recorded 5 minutes after each treatment. The volunteer profiles are shown in Table 3.

TABLE 3

| Volunteer | Gender | Age |
|---|---|---|
| 1 | Male | 42 |
| 2 | Male | 76 |
| 3 | Female | 25 |

The results of the study are shown in Table 4, below. These studies demonstrated that nebulization of sodium citrate did not result in any irritation or other negative side effects.

TABLE 4

| | | |
|---|---|---|
| Treatment | FEV1 | FVC |

Volunteer 1

| | | |
|---|---|---|
| No treatment | 4.02 | 4.17 |
| Water (0 Citrate) | 3.99 | 4.77 |
| 11 mM | 3.90 | 4.66 |
| 22 mM | 3.96 | 4.82 |

Volunteer 2

| | | |
|---|---|---|
| No treatment | 2.47 | 3.38 |
| Water (0 Citrate) | 2.28 | 3.54 |
| 11 mM | 2.39 | 3.48 |
| 22 mM | 2.28 | 3.40 |

Volunteer 3

| | | |
|---|---|---|
| No treatment | 2.68 | 2.75 |
| Water (0 Citrate) | 2.62 | 2.73 |
| 11 mM | — | — |
| 22 mM | 2.79 | 2.84 |

It will be appreciated that the methods and systems of the present invention may be embodied in a variety of different forms, and that the specific embodiments shown in the figures and described herein are presented with the understanding that the present disclosure is considered exemplary of the principles of the invention, and is not intended to limit the invention to the illustrations and description provided herein.

1-31. (canceled)

32. A method for treating or preventing a disorder characterized by the presence of biofilm in a subject in need thereof, the method comprising administering to the subject a composition comprising trisodium citrate and ammonium chloride in an amount effective to disrupt the biofilm compared to prior to administration, wherein the disorder is selected from the group consisting of: cystic fibrosis (CF), endocarditis, urinary tract infections, middle-ear infections, chronic sinusitis, chronic tonsillitis, gingivitis, periodontal disease, bronchiectasis, chronic obstructive pulmonary disease (COPD), asthma, neonatal meconium aspiration syndrome, smokers' cough, chronic tonsillitis, chronic vaginitis, and infections of implanted devices, such as catheters, heart valves, intrauterine devices and joint prostheses.

33. A method for treating or preventing a disorder characterized by the presence of biofilm in a subject in need thereof, the method comprising administering to the subject a composition comprising trisodium citrate and ammonium chloride in an amount effective to disrupt the biofilm compared to prior to administration, wherein the method is for treating or preventing a disorder in a patient following maxillofacial surgery and/or trauma.

34. The method of claim 33 wherein the method of treatment includes ventilation of patients with the medicament.

35. The method of claim 32, wherein the disorder is caused by a pathogen selected from the group consisting of: Haemophilus spp., such as Haemophilus influenza, Staphylococcus spp., such as Staphylococcus aureus, Pseudomonas spp., such as Pseudomonas aeruginosa, Burkholderia cepacia Complex, Wangiella dermatitidis, and Candida spp.

36. The method of claim 32, further comprising administering at least one therapeutic agent selected from the group consisting of: DNase, alginase, heparanase, antibiotics, and antimicrobial agents.

37. The method of claim 36, wherein the therapeutic agent is administered simultaneously to the composition.

38. The method of claim 37, wherein the therapeutic agent and the composition are administered simultaneously in a single formulation.

39. The method of claim 32, wherein the composition is administered in an aerosol form.

40. The method of claim 32, wherein the composition is administered to a target site selected from the group consisting of: respiratory tract, gastrointestinal tract and reproductive tract.

41. The method of claim 32, wherein the composition comprises between 10 mM to 80mM trisodium citrate and between 50 mM to 100 mM ammonium chloride.

42. The method of claim 41, wherein the composition comprises about 55 mM trisodium citrate and about 75 mM ammonium chloride.

43. A method for treating a bacterial or fungal infection in a patient in need thereof, the method comprising administering a composition comprising trisodium citrate and ammonium chloride, wherein the patient is afflicted with cystic fibrosis.

44. The method of claim 43, wherein the bacterial or fungal infection is caused by a pathogen selected from the group consisting of: Haemophilus spp., such as Haemophilus influenza, Staphylococcus spp., such as Staphylococcus aureus, Pseudomonas spp., such as Pseudomonas aeruginosa, Burkholderia cepacia Complex, Wangiella dermatitidis, and Candida spp.

45. The method of claim 43, further comprising administering at least one therapeutic agent selected from the group consisting of: DNase, alginase, heparanase, antibiotics, and antimicrobial agents.

46. The method of claim 43, wherein the composition is administered in an aerosol form.

47. The method of claim 43, wherein the composition is administered to a target site selected from the group consisting of: respiratory tract, gastrointestinal tract and reproductive tract.

48. The method of claim 43, wherein the composition comprises between 10 mM to 80mM trisodium citrate and between 50 mM to 100 mM ammonium chloride.

49. The method of claim 43, wherein the composition comprises about 55 mM trisodium citrate and about 75 mM ammonium chloride.

50. A composition comprising trisodium citrate and ammonium chloride in amounts effective to disrupt a biofilm in a patient when administered to a patient in need thereof compared to prior to administration of the composition, wherein the trisodium citrate and the ammonium chloride are the sole active ingredients in the composition.

51. The composition of claim 50, wherein the composition is formulated for delivery to the respiratory tract, the gastrointestinal tract and/or the reproductive tract.

52. The composition of claim 50, wherein the composition is isotonic and has a neutral pH.

53. A formulation comprising the composition of claim 50 and a therapeutic agent selected from the group consisting of: DNase, alginase, heparanase, antibiotics, ascorbic acid, and antimicrobial agents.

54. A composition comprising trisodium citrate and ammonium chloride in amounts effective to disrupt a biofilm in a patient when administered to a patient in need thereof compared to prior to administration of the composition, wherein the composition comprises between 10 mM to 80 mM trisodium citrate and between 50 mM to 100 mM ammonium chloride.

55. The composition of claim 54, wherein the composition comprises about 55 mM trisodium citrate and about 75 mM ammonium chloride.

56. The composition of claim 54, wherein the composition is isotonic and has a neutral pH.

57. A formulation comprising the composition of claim 54 and a therapeutic agent selected from the group consisting of: DNase, alginase, heparanase, antibiotics, ascorbic acid, and antimicrobial agents.

58. A composition consisting essentially of trisodium citrate and ammonium chloride, wherein the composition is formulated for administration to the respiratory tract, the gastrointestinal tract or the reproductive tract.

59. The composition of claim 58, wherein the trisodium citrate is present in an amount between 10 mM to 80 mM and the ammonium chloride is present in an amount between 50 mM to 100 mM ammonium chloride.

60. The composition of claim 58, wherein the trisodium citrate is present in an amount of about 55 mM trisodium citrate and the ammonium chloride is present in an amount of about 75 mM.

61. The composition of claim 58, wherein the composition is isotonic and has a neutral pH.
